SEQUENCE LISTING (10 sequences)

SEQ ID NO: 1 - DNA, 830 bp, Homo sapiens
  CDS (95)..(613)
cccccccgagcgccgctccggctgcaccgcgctcgctccgagtttcaggctcgtgctaagctagcgccgtcgtcgtctcccttcagtcgccatcatgattatctaccgggacctcatcagccacgatgagatgttctccgacatctacaagatccgggagatcgcggacgggttgtgcctggaggtggaggggaagatggtcagtaggacagaaggtaacattgatgactcgctcattggtggaaatgcctccgctgaaggccccgagggcgaaggtaccgaaagcacagtaatcactggtgtcgatattgtcatgaaccatcacctgcaggaaacaagtttcacaaaagaagcctacaagaagtacatcaaagattacatgaaatcaatcaaagggaaacttgaagaacagagaccagaaagagtaaaaccttttatgacaggggctgcagaacaaatcaagcacatccttgctaatttcaaaaactaccagttctttattggtgaaaacatgaatccagatggcatggttgctctattggactaccgtgaggatggtgtgaccccatatatgattttctttaaggatggtttagaaatggaaaaatgttaacaaatgtggcaattattttggatctatcacctgtcatcataactggcttctgcttgtcatccacacaacaccaggacttaagacaaatgggactgatgtcatcttgagctcttcatttattttgactgtgatttatttggagtggaggcattgtttttaagaaaaacatgtcatgtaggttgtctaaaaataaaatgcatttaaactcatttgagag

SEQ ID NO: 2 - protein, 172 aa, Homo sapiens
MIIYRDLISHDEMFSDIYKIREIADGLCLEVEGKMVSRTEGNIDDSLIGGNASAEGPEGEGTESTVITGVDIVMNHHLQETSFTKEAYKKYIKDYMKSIKGKLEEQRPERVKPFMTGAAEQIKHILANFKNYQFFIGENMNPDGMVALLDYREDGVTPYMIFFKDGLEMEKC

SEQ ID NO: 3 - DNA, 24 bp, Artificial
  Description of Artificial sequence Synthetic oligonucleotide
ccacaaccaccaagaactgcttag

SEQ ID NO: 4 - DNA, 21 bp, Artificial
  Description of Artificial sequence Synthetic oligonucleotide
gaaggggacgaaggaagagtg

SEQ ID NO: 5 - DNA, 23 bp, Artificial
  Description of Artificial sequence Synthetic oligonucleotide
gggaaatcgtgcgtgacgttaag

SEQ ID NO: 6 - DNA, 22 bp, Artificial
  Description of Artificial sequence Synthetic oligonucleotide
tgtgttggcgtacaggtctttg

SEQ ID NO: 7 - DNA, 29 bp, Artificial
  Description of Artificial sequence Synthetic oligonucleotide
ttggatccatgatcatctaccgggacctg

SEQ ID NO: 8 - DNA, 29 bp, Artificial
  Description of Artificial sequence Synthetic oligonucleotide
ttgaattcttaacatttctccatctctaa

SEQ ID NO: 9 - DNA, 28 bp, Artificial
  Description of Artificial sequence Synthetic oligonucleotide
gcgcagatctatgattatctaccgggac

SEQ ID NO: 10 - DNA, 28 bp, Artificial
  Description of Artificial sequence Synthetic oligonucleotide
ggccgaattcagatccaaaataattgcc

(19) United States
(12) Patent Application Publication
(10) Pub. No.: US 2007/0184485 A1
(43) Pub. Date: Aug. 9, 2007

(54) Method of diagnosing diseases relating to endometriosis

(76) Inventors: Yoshinori Kosugi, Tokyo (JP); Masahiko Kuroda, Tokyo (JP); Kosuke Oikawa, Tokyo (JP); Tetsuya Ohbayashi, Kyoto (JP)

Correspondence Address: WENDEROTH, LIND & PONACK, L.L.P., 2033 K STREET N. W., SUITE 800, WASHINGTON, DC, 20006-1021, US

(21) Appl. No.: 10/564,484

(22) PCT Filed: Jan. 13, 2004
(86) PCT No.: PCT/JP04/00160

§ 371 Date: Mar. 16, 2006

(30) Foreign Application Priority Data
Jul. 14, 2003 (JP) 2003-196459

(51) Int. Cl. G01N 33/53; C07K 16/22

(52) U.S. Cl. 435/7.1; 530/388.25

(57) ABSTRACT

The level of a histamine-releasing factor (HRF) protein in a biological sample of a subject is measured and the HRF protein level is compared with that of a normal biological sample. A significantly higher HRF protein level compared with that of the normal biological sample is used as an indicator of a disease related to endometriosis or the degree of its risk.

### TECHNICAL FIELD

The invention of this application relates to a molecular biological method of diagnosing a disease related to endometriosis. In addition, the invention of this application relates to a therapeutic drug and a therapeutic method for a disease related to endometriosis utilizing the molecular mechanism of the disease.

### BACKGROUND ART

Endometriosis is a common obstetrical and gynecological disease and affects 10% of all women in their reproductive years (non-patent document 1). A tissue of endometriosis goes through periodic proliferation and disintegration as eutopic endometrium, which causes periodic dysmenorrhea, dyspareunia, pelvic pain and hematuria during menstruation. Further, it has been reported that 30 to 40% of the infertility patients suffer from this disease (non-patent document 2). The mechanism in the transfer of an endometrial cell and the ectopical proliferation thereof in a part of patients is not known yet, however, there is a possibility that deregulation of an inflammatory cytokine may contribute to the progress of endometriosis (non-patent documents 3 and 4). In fact, activation of a monocyte and its intraperitoneal transfer are one of the immunologic abnormalities, which has been reported most consistently with regard to endometriosis (non-patent documents 5 to 8).

Dioxin is one of the endocrine disrupting chemicals land unevenly distributed in the environment. 3,3,7,8-tetrachlorodibenzo-p-dioxin (TCDD; dioxin) is a substance with the highest toxicity among dioxins and has a variety of toxic effects (e.g., immunotoxicity, hematotoxicity, teratogenicity, carcinogenicity and the like) (non-patent documents 9 and 10). The change in gene expression induced by TCDD and a related compound is triggered at the point where a toxin is bound to an arylhydrocarbon receptor (AhR), then a dimer is formed with an arylhydrocarbon receptor nuclear translocator (ARNT), and a complex which interacts with a gene regulation factor including an XRE (xenobiotic responsive element) motif is formed (non-patent documents 11 and 12). When monkeys were chronically exposed to TCDD, endometriosis was developed ranging in severity from mild to severe in a dose dependent manner (non-patent document 13). Therefore, several studies with regard to the correlation between dioxin and endometriosis were carried out (non-patent documents 14 to 18). However, the result that there is no correlation between TCDD exposure and endometriosis has been reported recently (non-patent documents 19 and 20), the correlation between dioxin exposure and endometriosis has remained unknown.

Incidentally, the inventors of this application have identified TCDD target genes including an IgE-dependent histamine-releasing factor (HRF) (non-patent documents 21 to 23). However, the correlation between an HRF as such a TCDD target gene product and endometriosis is not known at all.
Non-patent document 1: Wheeler J. M. J. Reprod Med. 1989, 34(1): 41-6
Non-patent document 2: Candiani G. B. et al. Obstet Gynecol. Surv. 1991, 46(6): 374-82
Non-patent document 3: Garcia-Velasco J. A. and Arici A. Fertil Steril. 1999, 71(6): 983-93
Non-patent document 4: Barcz et al. Med. Sci. Monit. 2000, 6(5): 1042-6
Non-patent document 5: Jolicoeur C. et al. Am. J. Pathol. 1998, 152(1): 125-33
Non-patent document 6: Lebovic D. I. et al. Fertil Steril 2001, 75(1): 1-10
Non-patent document 7: Hornung D. et al. Am. J. Pathol. 2001, 158(6): 1.949-54
Non-patent document 8: Blumenthal R. D. et al. Am. J. Pathol. 2000, 156(5): 1581-8
Non-patent document 9: Chapman D. E. and Schiller C. M. Toxicol Appl. Pharmacol. 1985, 78(1): 147-57
Non-patent document 10: McGregor D. B. et al. Environ Health Perspect. 1998, 106 Suppl 2: 755-60
Non-patent document 11: Sagawa K. and Fujui-Kuriyama T. J. Biochem. (Tokyo) 1997, 122(6): 1075-9
Non-patent document 12: Nebert D. W. Crit. Rev. Toxicol. 1989, 20(3): 153-74
Non-patent document 13: Rier S. E. et al. Fundam. Appl. Toxicol. 1993, 21(4): 433-41
Non-patent document 14: Gibbsons A. Science 1993, 262 (5183): 1373
Non-patent document 15: Obsteen K. G. and Sierra-Rivera E. Endocrinol 1997, 15(3): 301-8
Non-patent document 16: Bruner-Tran K. L. et al. Gynecol. Obstet. Invest. 1999, 48 Suppl. 1: 45-56
Non-patent document 17: Johson K. L. et al. Environ Health Perspect 1997, 105(7): 750-5
Non-patent document 18: Yang J. Z and Foster W. G. Toxicol. Ind. Health 1997, 13(1): 15-25
Non-patent document 19: Igarashi T. et al. Endocr. J. 1999, 46(6): 765-72
Non-patent document 20: Pauwels A. et al. Hum. Reprod. 2001, 16(10): 2050-5
Non-patent document 21: Oikawa K. et al. Cancer Res. 2001, 61(15): 5707-9
Non-patent document 22: Oikawa K. et al. Biochem. Biophys. Res. Commun. 2002, 290(3): 984-7
Non-patent document 23: Ohbayashi et al. FEBS Lett. 2001, 508(3): 341-4

### DISCLOSURE OF THE INVENTION

With regard to a diagnosis of endometriosis, there was no effective method other than an invasive method by abdominal endoscopy.

On the other hand, for a variety of human diseases, a molecular biological diagnosis using a marker protein specific to the disease or its gene expression as an indicator has become popular. This method does not require large-scale equipment and the physical strain to a subject is small, therefore, it is possible to make the diagnosis widely even for a lot of subjects who do not notice any symptoms. However, for endometriosis, an effective marker protein or a gene thereof for performing such a molecular biological diagnostic method has not been known.

The invention of this application has been made in view of the circumstances as described above, and an object of the invention is to provide a molecular biological diagnostic method or therapeutic method utilizing a marker closely related to endometriosis.

In addition, an object of the invention of this application is to provide various types of materials to be used in this diagnostic method or therapeutic method.

This application provides the following (1) to (14) inventions in order to solve the objects described above.
(1) A method of diagnosing a disease related to endometriosis, which comprises measuring the level of a histamine-releasing factor (HRF protein) in a biological sample from a subject, comparing the HRF protein level with that of a normal biological sample and determining that the subject showing a significantly higher HRF protein level compared with that of the normal biological sample is a patient with a disease related to endometriosis or a person with high risk thereof.
(2) An antibody recognizing an HRF protein.
(3) An antibody binding to an epitope different from the one to which an antibody of the invention (2) binds.
(4) The antibody of the invention (2) or (3), obtained by using, as an immunizing antigen, a peptide containing a sequence of 5 to 20 amino acid residues selected from the amino acid sequence at positions 90 to 130 of SEQ ID NO: 2.
(5) The antibody of the invention (2) or (3), obtained by using, as an immunizing antigen, a peptide containing a sequence of 5 to 20 amino acid residues selected from the amino acid sequence at positions 1 to 95 of SEQ ID NO: 2.
(6) The antibody of the invention (2) or (3), obtained by using, as an immunizing antigen, a peptide containing a sequence of 5 to 20 amino acid residues selected from the amino acid sequence at positions 115 to 172 of SEQ ID NO: 2.
(7) A method of diagnosing a disease related to endometriosis, which comprises at least the following steps of:
(a) contacting a biological sample from a subject with a support on which the antibody of the invention (2) has been immobilized;
(b) washing the support with which the biological sample has been contacted in the step (a);
(c) contacting the antibody of the invention (3), which has been labeled, with the support washed in the step (b);
(d) measuring a bound label or a free label on the support;
(e) comparing the label amount measured in the step (d), as an indicator of the HRF protein level, with the result of a normal biological sample; and
(f) employing a significantly higher HRF protein level compared with that of the normal biological sample as an indicator showing a disease related to endometriosis or the degree of its risk.
(8) A method of diagnosing a disease related to endometriosis, which comprises at least the following steps of:
(a) subjecting a biological sample from a subject to a treatment of tissue fixation;
(b) sectioning the fixed tissue specimen prepared in the step (a);
(c) subjecting the sectioned tissue obtained in the step (b) to immunohistological staining with the antibody of the invention (2);
(d) comparing the degree of the immunohistological staining by the step (c), as an indicator of the HRF protein level, with the result of a normal biological sample; and
(e) employing a significantly higher HRF protein level compared with that of the normal biological sample as an indicator showing a disease related to endometriosis or the degree of its risk.
(9) A kit for diagnosing a disease related to endometriosis comprising at least the antibody of the invention (2), which has been labeled.
(10) A kit for diagnosing a disease related to endometriosis comprising at least the following elements:
(a) the antibody of the invention (2); and
(b) the antibody of the invention (3), which has been labeled.
(11) A kit for diagnosing a disease related to endometriosis comprising at least the following elements:
(a) a support on which the antibody of the invention (2) has been immobilized; and
(b) an antibody of the invention (3), which has been labeled.
(12) An antibody recognizing an HRF protein and neutralizing the activity of the HRF protein.
(13) A therapeutic drug for a disease related to endometriosis, which comprises the antibody of the invention (12).
(14) A therapeutic method for a disease related to endometriosis, which comprises administering the antibody of the invention (12) or a therapeutic drug of the invention (13) into the body.

The inventors of this application investigated the expression of TCDD target genes (HRF and CYP1A1) in endometrial tissues and endometriotic implants. As a result, they found a high correlation between the progress of endometriosis and the HRF expression level, thus the invention of this application has been accomplished.

In this invention, a “disease related to endometriosis” means endometriosis, and dysmenorrhea, infertility, adenomyosis uteri and the like caused by endometriosis. “Diagnosis” means determination whether or not a subject suffers from a disease related to endometriosis, determination whether or not there is a risk of developing a disease related to endometriosis in future, and determination whether or not there is a risk of recurrence of a disease related to endometriosis after treatment. In addition, in the diagnosis, measurement of the degree of a developed disease related to endometriosis or its risk is included.

In this invention, an “HRF polynucleotide” means a polynucleotide (a molecule obtained by binding phosphate esters of a nucleoside (ATP, GTP, CTP and UTP; or dATP, dGTP, dCTP and dTTP) in which a purine or a pyrimidine has been bound to a sugar through a β-N-glycoside bond) encoding an HRF protein. Specifically, it is a genomic DNA encoding an HRF protein, an mRNA transcribed from the genomic DNA, a cDNA synthesized from the mRNA. In addition, it may be either a double strand or a single strand. Further. it may include a sense strand and an antisense strand of such a genomic DNA, mRNA or cDNA. In addition, a “polynucleotide” means a molecule in which 100 or more of the nucleotides described above have been bound together, and an “oligonucleotide” means a molecule in which 2 to 99 nucleotides have been connected together. Further, a “protein” and a “peptide” means a molecule composed of plural amino acid residues bound to each other through an amide bond (peptide bond). In particular, the one with 2 to 33 amino acid residues is referred to as an “oligopeptide” and the one with 34 or more of amino acid residues is referred to as a “polypeptide”.

In addition, with regard to the nucleotide sequences and amino acid sequences in the Sequence Listing, addition or deletion of one or more bases, replacement thereof with another base, or addition or deletion of one or more amino acid residues or replacement thereof with another amino acid based on such a base mutation is also included.

The other terms and concepts in this invention will be defined in detail in the description of the embodiments or Examples of the invention. The terms are basically in accordance with IUPAC-IUB Commission on Biochemical Nomenclature or based on the meanings of terms used commonly in the art. In addition, various techniques used for implementing this invention can be easily and surely carried out by those skilled in the art based on known literatures and the like except for the techniques whose sources are particularly specified. For example, preparation of a drug can be carried out in accordance with the methods described in Remingtone's Pharmaceutical Sciences, 18th Edition, ed. A. Gennaro, Mack Publishing Co., Eastout, Pa., 1990, and techniques of genetic engineering and molecular biology can be carried out in accordance with the methods described in J. Sambrook, E. F. Fritsch & T. Maniatis, “Molecular Cloning: A Laboratory Manual (2nd edition)”, Cold Spring Harbor Laboratory Press, Cold Spring Harbor, N.Y. (1989); D. M. Glover et al. ed., “DNA Cloning”, 2nd ed., Vol. 1 to 4, (The Practical Approach Series), IRL Press, Oxford University Press (1995); Ausubel, F. M. et al., Current Protocols in Molecular Biology, John Wiles & Sons, New York, N.Y., 1995; Japanese Biochemical Society ed., “Zoku Seikagaku Jikken Koza 1, Idenshi Kenkyuho II” Tokyo Kagaku Dozin (1986); Japanese Biochemical Society ed., “Shin Seikagaku Jikken Koza 2, Kakusan III (Krumikae DNA Gijutsu)” Tokyo Kagaku Dozin (1992); R. Wu ed., “Methods in Enzymology”, Vol. 68 (Recombinant DNA), Academic Press, New York (1980); R. Wu et al. ed., “Methods in Enzymology”, Vol. 100 (Recombinant DNA, Part B) & 101 (Recombinant DNA, Part C), Academic Press, New York (1983); R. Wu et al. ed., “Methods in Enzymology”, Vol. 153 (Recombinant DNA, Part D), 154 (Recombinant DNA, Part E) & 155 (Recombinant DNA, Part F), Academic Press, New York (1987); J. H. Miller ed, “Methods in Enzymology”, Vol. 204, Academic Press, New York (1991); R. Wu et al. ed., “Methods in Enzymology”, Vol. 218, Academic Press, New York (1993); S. Weissman (ed.), “Methods in Enzymology”, Vol. 303, Academic Press, New York (1999); J. C. Glorioso et al. (ed.), “Methods in Enzymolgy”, Vol. 306, Academic Press, New York (1999), etc. or the methods described in the references cited therein or substantially the same methods of modifications thereof (the description therein is included in the disclosure of this description by referring to it).

### BRIEF DESCRIPTION OF DRAWINGS

FIG. 1 shows the results of investigating the expression of HRF and CYP1A1 in normal endometrial tissues, eutopic endometrial tissues derived from a patient with endometriosis and endometriotic implants. (A) shows the mRNA level of HRF investigated by Northern blot analysis. The blot was reprobed by using a human β-actin probe, and the total RNA level was determined. The mRNA level of CYP1A1 in a sample investigated by Northern blot was determined by quantitative RT-PCR using Southern blot analysis. In order to confirm the accuracy of quantification, investigation was carried out by using different concentration (5-fold) of cDNA samples (1× and 5×) as a PCR template in the same position. β-actin was used as an internal control for mRNA level. (B) shows the image displays for the mRNA levels of HRF and CYP1A1 in the same manner. The mRNA levels were normalized to β-actin signals using a densintometry (MOLECULAR IMAGER, Nippon Bio-Rad). The sample 11-2A indicates the mRNA level of HRF and 10-2A indicates the mRNA level of CYP1A1, which were optionally defined as 10. In the case where plural samples were derived from one individual, the mean value was calculated and shown. Error bars indicate the maximum values of plural samples. 12-1, 7-1, 8-1 and 6B correspond to normal endometrial tissues and 1C marked with an asterisk corresponds to eutopic endometrium of a patient with endometriosis.

FIG. 2 shows the results of investigating the expression of HRF in endometriotic implants. (A) shows the results of the Northern blot analysis of HRF expression in normal endometrial tissues, eutopic endometrial tissues of a patient with endometriosis and endometriotic implants. The blot was reprobed by using a human 62-actin probe, and the total RNA level was determined. N, Eu and En on the columns indicate normal endometrial tissues, eutopic endometrial tissues of a patient with endometriosis and endometriotic implants, respectively. (B) is a graph showing the mRNA levels of HRF measured by Northern blot analysis with regard to the samples investigated in FIG. 1A and FIG. 2A. The mRNA levels of HRF were normalized to β-actin signals using a densintometry (MOLECULAR IMAGER, Nippon Bio-Rad). The mRNA level of the sample 6B was optionally defined as 1. In the case where plural samples were derived from one individual, the mean value was calculated and shown. Error bars indicate the maximum values of plural samples.

FIG. 3 shows the results of immunohistochemical analysis of the expression of HRF and CD68. A positive part is visualized by brown staining. Hematoxylin was used for reverse staining. (A) and (B) show the detection of an HRF protein in a normal endometrial tissue (A: Proliferative phase, B: Secretory phase, magnification of the: original image: ×200). (C) shows the detection of an HRF protein in the inside of an uterine endometriotic implant (magnification of the original image: ×200). (D) shows the hematoxylin-eosin staining of a series of sections showing a form of an endometriotic implant (magnification of the original image: ×200). (E) shows the detection of an HRF protein in the same visual field as (C) at a larger magnification (magnification of the original image: ×400). (F) shows the immunohistochemical localization of CD68-positive macrophage in a series of sections of an endometriotic implant (magnification of the original image: ×400).

FIG. 4 shows the results of transplantation assay. (A) shows the results of Western blot analysis of HRF proteins in NIH3T3 cells. “wt” corresponds to a parent NIH3T3 cell, “HRF” corresponds to a cell strain (pMSCV-HRF-3T3) stably expressing HRF after infected with a retrovirus vector containing HRF, and “vector” corresponds to a control cell (pMSCV-3T3) infected with an empty vector. (B) shows a high transplantation ratio exhibited by an HRF-overexpressing cell in a nude mouse. The marks on the vertical axis indicate the following conditions. “+++” indicates the condition where a large number of transplanted colonies were observed. “++” indicates the condition where several tens of transplanted colonies were observed. “+” indicates the condition where several transplanted colonies were observed. “−” indicates the condition where no implanted colony was observed. The individual mice injected with a control cell or an HRF-overexpressing cell are indicated by an open circle and a closed circle, respectively.

### BEST MODE FOR CARRYING OUT THE INVENTION

A diagnostic method according to the invention (1) of this application is a method of measuring the level of a histamine-releasing factor (HRF protein) from a biological sample of a subject, and diagnosing a disease related to endometriosis by using this HRF protein level as an indicator. In other words, it is determined that a subject showing a significantly higher HRF protein level compared with that of a normal biological sample is a patient with a disease related to endometriosis or a person with high risk thereof. That is, it is determined that a subject showing a significantly high level of an existing HRF protein is a patient with a disease related to endometriosis or a person with high risk thereof. The level of an existing HRF protein expressed from an HRF gene is closely related to a disease related to endometriosis, therefore, a diagnosis of endometriosis can be made by using this HRF protein level in a biological sample (e.g., an endometrial tissue or the like) of a subject as an indicator. In addition, a “significantly higher” HR protein level means the case where the HRF protein level in a subject is higher than the HRF protein level measured in an normal biological sample (i.e., a biological sample of a healthy subject) by 10% or more, preferably by 30% or more, more preferably by 70% or more, and most preferably by 100% or more. Further, this “significantly higher” means the case where, for example, when a mean value of the expression levels of HRF polynucleotides in plural samples from the same subject and a similar mean value in plural normal samples are statistically examined, the former is significantly higher than the latter.

The diagnostic method of the invention (1) in which the HRF protein level is used as an indicator as described above can be carried out in accordance with known techniques of genetic engineering and molecular biology by detecting and measuring the HRF protein level by a method known in the art for detecting and measuring the level of a specific protein, for example, in situ hybridization, Western blotting, a variety of immunohistological methods and the like. A measuring system for the HRF protein level, a detection system for a disease related to endometriosis and a risk detection system for a disease related to endometriosis which utilize such a technique, a reagent, a method, a process, and an analytical program, which are used for the systems, are all included in the techniques of this invention and systems to be used therefor.

This application provides, as a material to be used in the foregoing diagnostic method of the invention (1), particularly the following antibodies of the inventions (2) and (3).

The antibody of the invention (2) is an antibody specifically recognizing an HRF protein (anti-HRF antibody). Note that the term “antibody” herein may be the one to be used in the extensive meaning, a single monoclonal antibody to a desired HRF polypeptide or a peptide fragment related thereto, or an antibody composition having specificity to a variety of epitopes. In addition, it includes a monovalent antibody, a polyvalent antibody, a polyclonal antibody and a monoclonal antibody, and also represents a natural (intact) molecule, a fragment thereof and a derivative thereof, and includes fragments such as F(ab′)₂, Fab′ and Fab. Further, it may include a chimera antibody or a hybrid antibody having binding sites for at least two antigens or epitopes, a recombinant antibody with dual specificity such as quadrome or triome, an interspecies hybrid antibody, an antiidio type antibody, the one which has been chemically modified or processed and is considered to be a derivative thereof, an, antibody obtained by applying a known cell fusion, hybridoma technique or antibody engineering, or using a synthetic or semisynthetic technique, an antibody prepared by applying a known conventional technique in view of the production of antibody or using a DNA recombinants technique, an antibody having a neutralizing property related to a target antigenic substance or a target epitope described and defined in this description, and an antibody having a binding property. A particularly preferred antibody is the one which can specifically distinguish a natural HRF protein level (polypeptide), and examples thereof include the foregoing antibodies of the inventions (4) to (6) and the like.

In other words, the antibodies of the inventions (4) to (6) are antibodies prepared by using a partial peptide of an HRF protein composed of the amino acid sequence of SEQ ID NO: 2 as an antigen, respectively, and are antibodies recognizing different sites of the HRF protein, respectively. The HRF peptide for preparing such an antibody is synthesized by, for example, the Fmoc-bop method with, for example, a peptide synthesizer. A cysteine may be introduced into the N-terminus of the HRF peptide. The synthesized peptide is purified by high performance liquid chromatography using a μBondasphere, a C18 column (Waters) and the like and used as an immunizing antigen.

The antibody of the invention (3) is an antibody, which binds to an epitope different from the one to which the foregoing antibody of the invention (2) binds. Such an antibody is prepared as a similar polyclonal antibody or monoclonal antibody to the one described above by using, as an immunogen, a fragment different from the oligopeptide for preparing the foregoing antibody of the invention (2). For example, among the foregoing antibodies of the inventions (4) to (6), any one of them becomes an antibody of the invention (2), and any one of the others becomes an antibody of the invention (3).

Such an antibody can be obtained, for example in the case of a polyclonal antibody, from serum after immunizing an animal with an HRF protein or a partial fragment (oligopeptide) thereof as an immunogen. Alternatively, it can be prepared by introducing a recombinant vector of an HRF protein polynucleotide into the muscle or the skin of an animal with an injector or a gene gun, and collecting the serum. As the animal, mouse, rat, hamster, rabbit, goat, sheep, cow, horse, pig, dog, cat, monkey, chicken or the like is used. Further, in some cases, it is preferred that the animal should be selected considering the compatibility with a parent cell to be used for cell fusion.

Immunization of an animal with a sensitizing antigen is carried out in accordance with a known method, for example, it can be carried out in accordance with the method described in Shigeru Muramatsu et al. ed., Jikken Seibutsugaku Koza 14, Immunobiology, Maruzen, 1985; Japanese Biochemical Society ed., Zoku Seikagaku Jikken Koza 5, Meneki Seikagaku Kenkyuho, Tokyo Kagaku Dozin, 1986; Japanese Biochemical Society ed., Shin Seikagaku Jikken Koza 12, Molecular Immunology III, Antigens, Antibodies and Complements, Tokyo Kagaku Dozin, 1992; or the like. For example, as a general method, immunization is carried out by injecting a sensitizing antigen intraperitoneally or subcutaneously into a mammal or the like. In addition, during the immunization with the sensitizing antigen, an appropriate carrier can be also used. Immunization is attained by injecting an immunizing agent (if necessary, together with an adjuvant) once or more times into a mammal. Typically, the immunizing agent and/or an adjuvant is subcutaneously or intraperitoneally injected into a mammal plural times. As the immunizing agent, the one including the foregoing antigen peptide or a peptide fragment related thereto can be exemplified. The immunizing agent may be used in the form of a conjugate with a protein (e.g., one of the foregoing carrier proteins) known to be antigenic in the mammal to be treated for immunization. Examples of the adjuvant include, for example, Freund's complete adjuvant, Ribi adjuvant, pertussis vaccine, BCG, lipid A, liposomes, aluminum hydroxide, silica and the like.

An antiserum containing the polyclonal antibody can be prepared from the blood collected from the animal after feeding the immunized animal for a predetermined period. After confirming that the obtained antiserum recognizes HRF, it is submitted to use as a predetermined active ingredient of this invention.

As the anti-HRF antibody of this invention, the one obtained as a monoclonal antibody derived from a mammal can be also used. The monoclonal antibody prepared against the antigenic substance can be produced by any of the methods capable of providing the production of antibody molecules in a series of cell lines under cultivation. The modifier “monoclonal” indicates the characteristic of an antibody that it is obtained from a substantially homogeneous antibody population. It is not to be construed that the antibody should be produced by a certain specific method. Individual monoclonal antibodies include a population of the same antibodies except that a slight amount of a mutant possibly formed spontaneously may be present therein. Monoclonal antibodies have high specificity and are directed to one single antigenic site. As compared with an ordinary (polyclonal) antibody preparation typically containing various antibodies directed to different antigenic determinants (epitopes), each monoclonal antibody is directed to one single antigenic determinant on the antigen. In addition to their specificity, monoclonal antibodies are synthesized by hybridoma culture and are superior in that they are not or only a little contaminated with other immunoglobulins. The monoclonal antibodies include hybrid antibodies and recombinant antibodies. They can be obtained by replacing a variable region domain thereof with a constant region domain or replacing a light chain thereof with a heavy chain, replacing a chain derived from a certain species with a chain derived from another species, or fusing them with a heterogeneous protein irrespective of their origin or immunoglobulin class or subclass as long as they show a desired biological activity (e.g. U.S. Pat. No. 4,816,567; Monoclonal Antibody Production Techniques and Applications, pp. 79-97, Marcel Dekker, Inc., New York, 1987, etc).

In addition, the monoclonal antibody can be prepared in accordance with a known method for preparing monoclonal antibodies (“Monoclonal Antibody”, co-authored by Komei Nagamune and Hiroshi Terada, Hirokawa Shoten, 1990; “Monoclonal Antibody”, James W. Goding, third edition, Academic Press, 1996).

An HRF protein or HRF peptide for preparing such an antibody can be obtained by, for example, a known in vitro transcription/translation method of employing a recombinant expression vector containing an HRF polynucleotide, or a gene recombinant technique using an appropriate host (a prokaryotic cell such as E. coli or Bacillus subtilis, yeast, an insect cell, a cell of an animal or a plant (including, for examples an insect such as silk worm), etc.)—vector system (including also, for example, a baculovirus vector system). For example, based on the HRF gene/amino acid sequence of SEQ ID NO: 1 in the sequence listing, a gene sequence encoding HRF or a domain of a part thereof, a protein or a polypeptide fragment of a part of HRF or a peptide with a partial amino acid sequence corresponding to the amino acid sequence of HRF is inserted into a known expression vector system, whereby an appropriate host cell is transformed, then the target HRF protein or domain protein of a part thereof, protein or polypeptide fragment of a part of HRF or peptide with a partial amino acid sequence corresponding to the amino acid sequence of HRF is purified from the host cell or the culture supernatant by a known method. In addition, particularly, an oligopeptide can be chemically synthesized by a known method such as the solid phase method.

Incidentally, with regard to the HRF polynucleotide, a variety of mutants (e.g., GenBank/XM_{—}294045, XM_{—}038391, XM_{—}293291, XM_{—}209741, XM_{—}210566, XM_{—}066706, XM_{—}066675, XM_{—}071321, etc.) are known. HRF cDNA shown in SEQ ID NO. 1 (nucleotide sequence) (or TPT-1: GenBank/NM_{—}003295) is exemplified as a preferred one. Such a polynucleotide can be easily obtained by a known method, respectively. For example, in the case of a cDNA, it can be obtained by synthesizing a cDNA library by using a known method (Mol. Cell Biol. 2, 161-170, 1982; J. Gene 25, 263-269, 1983; Gene, 150, 243-250, 1994), and by a method of isolating the respective cDNAs with the use of a probe DNA prepared based on the known nucleotide sequences, respectively. The obtained cDNA can be amplified by a commonly performed gene amplification method such as the PCR (polymerase chain reaction) method, NASBN (nucleic acid sequence based amplification) method, TMA (transcription-mediated amplification) method, or SDA (strand displacement amplification) method. In addition, by using a primer set provided by this invention, a necessary amount of each cDNA can be obtained by also the RT-PCR method in which mRNA isolated from a human cell is used as a template.

In addition, the HRF oligonucleotide encoding a specific HRF peptide can be obtained by, for example, digesting the foregoing polynucleotide (DNA) with an appropriate restriction enzyme. Alternatively, it can be synthesized in vitro by a known chemical synthesis technique as described in Carruthers (1982) Cold Spring Harbor Symp. Quant. Biol. 47: 411-418; Adams (1983) J. Am. Chem. Soc. 105: 661; Belousov (1997) Nucleic Acid Res. 25: 3440-3444; Frenkel (1995) Free Radic. Biol. Med. 19: 373-380; Blommers (1994) Biochemistry 33: 7886-7896; Narang (1979) Meth. Enzymol. 68: 90; Brown (1979) Meth. Enzymol. 68: 109; Beaucage (1981) Tetra. Lett. 22: 1859; and U.S. Pat. No. 4,458,066.

The antibodies of the inventions (2) and (3) are used after they are further purified if necessary. As the method for purifying and isolating the antibody, a conventionally known method, for example, salting out such as the ammonium sulfate precipitation, gel filtration using Sephadex or the like, ion exchange chromatography, electrophoresis, dialysis, ultrafiltration, affinity chromatography, high-performance liquid chromatography or the like can be used for purification. Preferably, ascitic fluid containing the antiserum or the monoclonal antibody can be purified and isolated by ammonium sulfate fractionation, followed by treatment with an anion exchange gel such as DEAE-Sepharose, and an affinity column such as a protein A column. Especially preferred are affinity chromatography with an immobilized antigen or antigen fragment (e.g., synthetic peptide, recombinant antigen protein or peptide, site specifically recognized by the antibody), affinity chromatography with an immobilized protein A, hydroxyapatite chromatography and the like.

By treatment of those antibodies with an enzyme such as trypsin, papain or pepsin, antibody fragments such as Fab, Fab′ and F(ab′)₂, if necessary followed by reduction may also be used. The antibodies can be used in any of the known assay methods, for example, competitive binding assay, direct and indirect sandwich assay, and immunoprecipitation assay (Zola, Monoclonal Antibodies: A Manual of Techniques, pp. 147-158 (CRC Press, Inc. 1987)).

In order to conjugate the antibody to a detectable atomic group, any of the methods known in the art can be used, and examples thereof include, for example, the methods described in David et al., Biochemistry, Vol. 13, pp. 1014-1021 (1974); Pain et al., J. Immunol. Meth., 40: pp. 219-231 (1981); and “Methods in Enzymology”, Vol. 184, pp. 138-163 (1990). As the antibody to be provided with a labeling substance, an IgG fraction and, further. the specific binding site Fab′ which can be obtained by reduction after pepsin digestion can be used.

A large number of supports capable of immobilizing an antigen or antibody are known, and an appropriate one can be used by selecting it from among them in this invention. Various supports to be used in an antigen-antibody reaction or the like are known and, of course, an appropriate one can be used by selecting it from such known supports also in this invention. Especially preferred for use are, for example, glass, activated glass such as aminoalkylsilylated glass, porous glass, silica gel, silica-alumina, alumina, magnetized iron, magnetized alloys and other inorganic materials, polyethylene, polypropylene, polyvinyl chloride, polyvinylidene fluoride, polyvinyl polymers, polyvinyl acetate, polycarbonates, polymethacrylates, polystyrene, styrene-butadiene copolymers, polyacrylamide, crosslinked polyacrylamide, styrene-methacrylate copolymers, polyglycidyl methacrylate, acrolein-ethylene glycol dimethacrylate copolymers and the like, crosslinked albumin, collagen, gelatin, dextran, agarose, crosslinked agarose, cellulose, microcrystalline cellulose, carboxylmethylcellulose, cellulose acetate and other natural or modified cellulose, crosslinked dextran, nylons and other polyamides, polyurethanes, polyepoxy resins and other organic polymers, polymers obtained by emulsion polymerization thereof, silicone rubbers and the like, cells, erythrocytes and the like. If necessary, they may have a functional group introduced therein using a silane coupling agent.

Examples of the support include particles, minute particles, microparticles, membrane, filter paper, beads, tubes, cuvettes, inside walls of test vessels, for example, test tubes, titer plates, titer wells, microplates, glass cells, cells made of a synthetic material such as synthetic resin cells, and the surfaces of solid substances (bodies) such as glass rods, rods made of a synthetic material, rods having a thickened or tapered end, rods having a round projection or flat projection at an end, and thin plate-like rods.

The binding of the anti-HRF antibody with such a support can be attained by physical means such as adsorption, by chemical means using a condensing agent or an activated form, by means utilizing a mutual chemical binding reaction or the like.

The antibodies of the inventions (2) and (3) include antibodies labeled with a labeling substance, respectively. Examples of the labeling substance include enzymes, enzyme substrates, enzyme inhibitors, prosthetic groups, coenzymes, enzyme precursors, apoenzymes, fluorescent substances, dye substances, chemoluminescent compounds, luminescent substances, chromophores, magnetic substances, metal particles such as gold colloid, nonmetallic element particles such as selenium colloid, radioactive substances, and the like. As a preferred labeling substance, an enzyme, a chemical substance including a radioisotope or a fluorescent dye can be used. There is no particular restriction on the enzyme as long as it fulfills the requirement such as a large turnover number, stability even upon binding to an antibody and an ability of specifically staining a substrate, and an enzyme to be used in common EIA can be used. Examples of the enzyme can include dehydrogenases, reductases, oxidases and other oxidation-reduction enzymes, transferases catalyzing the transfer of, for example, an amino group, carboxyl group, methyl group, acyl group or phosphoryl group, for example, hydrolases hydrolyzing an ester bond, glycoside bond, ether bond or peptide bond, such as lyases, isomerases, ligases and the like. A plurality of enzymes may be used in combination for detection purposes. For example, enzymatic cycling can be also used. The enzyme label or the like can be also replaced with a biotin label and an enzyme-labeled avidin (streptavidin). In this way, it is possible to suitably employ a sensitivity increasing method known in the art, for example, the use of a biotin-avidin system or the use of a secondary antibody such as an antibody to an anti-HRF antibody. It is also possible to use a plurality of different types of labels. In such a case, it is also possible to carry out a plurality of measurements continuously or discontinuously, and simultaneously or separately.

Typical examples of the enzyme label include peroxidases such as horseradish peroxidase, galactosidases such as E. coli-derived β-D-galactosidase, malate dehydrogenase, glucose-6-phosphate dehydrogenase, glucose oxidase, glucoamylase, acetylcholine esterase, catalase, bovine small intestine-derived alkaline phosphitase, alkaline phosphatases such as E. coli-derived alkaline phosphatase and the like.

The conjugation of such an enzyme with the antibody can be carried out by a known method of employing a crosslinking agent such as a maleimide compound. As the substrate, a known substance can be used according to the type of an enzyme to be used, and examples thereof include umbelliferone derivatives such as 4-methylumbelliferyl phosphate, phosphorylated phenol derivatives such as nitrophenyl phosphate and the like. For example, in the case where peroxidase is used as an enzyme, 3,3′,5,5′-tetramethylbenzidine can be used, and in the case where alkaline phosphatase is used as an enzyme, p-nitrophenol or the like can be used. In this invention, the combination of enzyme-reagents may also be used for the formation of signals, for example the combination of 4-hydroxyphenylacetic acid, o-phenylenediamine (OPD), tetramethylbenzidine (TMB), 5-aminosalicylic acid, 3,3-diaminobenzidine tetrahydrochloride (DAB), 3-amino-9-ethylcarbazole (AEC), tyramine, luminol, lucigenin luciferin or a derivative thereof, Pholad luciferin or the like with peroxidase such as horseradish peroxidase, the combination of Lumigen PPD, (4-methyl)umbelliferyl phosphate, p-nitrophenol phosphate, phenol phosphate, bromochloroindolyl phosphate (BCIP), AMPAK™ (DAKO), AmpliQ™ (DAKO) or the like with alkaline phosphatase, the combination of an umbelliferyl galactoside such as 4-methylumbelliferyl-β-D-galactoside, a nitrophenyl galactoside such as o-nitrophenol-β-D-galactoside or the like with β-D-galactosidase or glucose-6-phosphate dehydrogenase, and the combination of ABTS with glucose oxidase, and a compound which can form a quinol compound such as hydroquinone, hydroxybenzoquinone or hydroxyanthraquinone, a thiol compound such as lipoic acid or glutathione, a phenol derivative, a ferrocene derivative or the like under the action of an enzyme or the like can be used.

As the radioisotope, the one used in a common RIA such as ³²P, ¹²⁵I, ¹⁴C, ³⁵S or ³H can be used. Examples of the fluorescent substance or chemiluminescent compound include fluorescein isothiocyanate (FITC), rhodamine derivatives such as rhodamine B isothiocyanate, tetramethylrhodamine isothiocyanate (RITC) and tetramethylrhodamine isothiocyanate isomer R (TRITC), 7-amino-4-coumarin-3-acetic acid, dansyl chloride, dansyl fluoride, fluorescamine, phycobilin protein, acridinium salts, lumiferin, luciferase, aequorin and other luminols, imidazole, oxalate esters, rare earth chelate compounds, coumarin derivatives and the like. As the fluorescent dye, the one used for a common fluorescent antibody method can be used. For detecting the resulting signal including coloring, fluorescence and the like, visual observation may be employed, or a known apparatus may also be used, thus, for example, a fluorophotometer or a plate reader can be used. For detecting the signal emitted by a radioisotope (isotope) or the like, a known apparatus may be used, for example, a gamma counter or scintillation counter or the like may also be used.

The labeling of antibody can be carried out by utilizing the reaction between a thiol group and a maleimide group, the reaction between a pyridyl disulfide group and a thiol group, the reaction between an amino group and an aldehyde group or the like, and an appropriate method can be applied by selecting it from among known methods, methods that can be easily carried out by those skilled in the art and, further modifications thereof. A condensing agent which can be used in preparing the immunogenic conjugate, a condensing agent which can be used in binding to the support or the like can be used. Examples of the condensing agent include, for example, formaldehyde, glutaraldehyde, hexamethylene diisocyanate, hexamethylene diisothiocyanate, N,N′-polymethylenebis-iodoacetamide, N,N′-ethylenebismaleimide, ethylene glycol bissuccinimidyl succinate, bisdiazobenzidine, 1-ethyl-3-(3-dimethylaminopropyl)carbodiimide, succinimidyl3-(2-pyridyldithio)propionate(SPDP), N-succinimidyl4-(N-maleimidomethyl)cyclohexane-1-carboxylate (SMCC), N-sulfosuccinimidyl4-(N-maleimidomethyl)cyclohexane-1-carboxylate, N-succinimidyl(4-iodoacetyl)aminobenzoate, N-succinimnidyl 4-(1-maleimidophenyl)butyrate, N-(ε-maleimidocaproyloxy)succinimide(EMCS), iminothiolane, S-acetyl-mercaptosuccinic acid anhydride, methyl3-(4′-dithiopyridyl)propionimidate, methyl4-mercaptobutyrylimidate, methyl3-mercapto-propionimidate, N-succinimidyl S-acetylmercaptoacetate and the like.

One aspect in the diagnostic method of employing such an antibody is a method for detecting the binding of the antibody to an HRF protein in a liquid phase system. For example, a labeled antibody obtained by labeling an antibody of the invention (2) is brought into contact with a biological sample to bind the labeled antibody to the HRF protein, and this conjugate is separated. The separation can be carried out by a method of separating the conjugate of the HRF protein and the labeled antibody by a known separation method (chromatography, solid phase method or the like), etc. In addition, a method in accordance with the known Western blot method can be adopted. With regard to the measurement of the labeled signal, in the case of using an enzyme as the label, a substrate which develops color by being decomposed due to an enzymatic action is added, the activity of the enzyme is obtained by optically measuring the amount of decomposed substrates, which is converted into the amount of bound antibodies, and the amount of antibody is calculated in comparison with the standard value. In the case of using a radioisotope, the amount of radiation emitted by the radioisotope is measured with a scintillation counter or the like. In addition, in the case of using a fluorescent dye, the fluorescent amount may be measured with a measuring apparatus combined with a fluorescence microscope.

In another diagnostic method in the liquid phase system, an antibody (primary antibody) of the invention (2) is brought into contact with a biological sample to bind the primary antibody to an HRF protein, a labeled antibody (secondary antibody) of the invention (2) is bound to this conjugate, and the labeled signal in the conjugate of the third party is detected. In addition, in order to further enhance the signal, first a non-labeled secondary antibody is bound to the conjugate of an antibody and an antigen peptide, and a labeling substance may be conjugated to this secondary antibody. Such conjugation of the labeling substance to the secondary antibody can be carried out by, for example, biotinylating the secondary antibody and avidinylating the labeling substance. In addition, an antibody (tertiary antibody) that recognizes a partial region of the secondary antibody (e.g., Fc region) is labeled, and the tertiary antibody may be bound to the secondary antibody. Note that for both of the primary antibody and the secondary antibody, monoclonal antibodies can be used, or for either of the primary antibody or the secondary antibody, a polyclonal antibody can be used. The separation of the conjugate from the liquid phase or the detection of the signal can be carried out in the same manner as described above. In addition, a diagnostic kit of the invention (10) is provided as the one capable of performing such a diagnostic method conveniently and in a wide range.

Another diagnostic method of employing the antibody is a method of testing the binding of the antibody to the HRF protein in a solid phase system. This method in the solid phase system is a preferred method due to the detection of a very little amount of the HRF protein and the convenience of the operation. More specifically, this method in the solid phase system is a method in which an antibody of the invention (2) is immobilized on a resin plate, membrane or the like, an HRF protein is bound to this immobilized antibody, a non-bound protein is washed out, a labeled antibody obtained by labeling an antibody of the invention (3) is bound to the conjugate of the antibody and the HRF protein remaining on the plate, then the signal of this labeled antibody is detected. This method is what is called a “sandwich method”, and in the case of using an enzyme as a marker, it is a widely used method as “ELISA (enzyme linked immunosorbent assay)”. With regard to the two types of antibodies, monoclonal antibodies can be used for both antibodies, or a polyclonal antibody can be used for either of them.

The diagnosis in this invention can be made by immunostaining, for example tissue or cell staining, immune electron microscopy, or immunoassay, for example competitive immunoassay or noncompetitive immunoassay, and radioimmunoassay (RIA), fluoroimmunoassay (FIA), luminescent immunoassay (LIA), enzyme immunoassay (EIA), ELISA or the like can be also used. B-F separation may be performed, or the assay can be performed without such separation. Preferred are RIA, EIA, FIA, LIA, and further, sandwich assay. The sandwich assay may include simultaneous sandwich assay, forward sandwich assay, reversed sandwich assay and the like.

As the assay system for HRF protein level in the invention of this application, for example, a protein assay system such as immunostaining or immune electron microscopy for a tissue, a protein assay system such as EIA, RIA, FIA, LIA or Western blotting for a tissue extract, blood, body fluid or the like can be carried out.

In the assay system of EIA, for example, in the case of the competitive method, the anti-HRF antibody is used as an immobilized antibody and a labeled antigen and an unlabeled antigen (an HRF protein or a fragment peptide thereof and the like are exemplified as the antigen) are used and, in the case of the noncompetitive method, for example the sandwich method, an immobilized anti-HRF antibody or a labeled anti-HRF antibody can be used or the anti-HRF antibody may be directly labeled or an antibody to the anti-HRF antibody may be labeled without immobilization or with immobilization. As the sensitivity increasing method, examples thereof include in the combination—with a non-enzyme-labeled primary antibody, a method of employing a macromolecular polymer and an enzyme and the primary antibody (application of Envision reagent: Enhanced polymer one-step staining (EPOS)), and in the combination with a non-enzyme-labeled secondary antibody, for example, the combination of an enzyme and an anti-enzyme antibody complex in the PAP (peroxidase-antiperoxidase) method or the like, the combination of a biotin-labeled secondary antibody and a biotin-labeled enzyme-avidin complex in the SABC (avidin-biotinylated peroxidase complex) method or the like, the combination of a biotin-labeled secondary antibody and a biotin-labeled enzyme-streptavidin complex in the ABC (streptavidin-biotin complex) method, the LSAB (labeled streptavidin-biotin) method or the like, the combination of SABC, a biotin-labeled tyramide and an enzyme-labeled streptavidin in the CSA (catalyzed signal amplification) method, and a method of employing a secondary antibody and an enzyme labeled with a macromolecular polymer and the like.

For the details of such a general technical means, reference may be made to reviews, reference books and the like (e.g., the description in Hiroshi Irie (ed.), “Radioimmunoassay” (published by Kodansha, 1974); Hiroshi Irie (ed.), “Radioimmunoassay; Second Series” (published by Kodansha, 1979); Eiji Ishikawa, et al. (ed.), “Enzyme Immunoassay” (published by Igaku Shoin, 1978); Eiji Ishikawa, et al. (ed.), “Enzyme Immunoassay” (Second Edition) (published by Igaku Shoin, 1982); Eiji Ishikawa, et al. (ed.), “Enzyme Immunoassay” (Third Edition) (published by Igaku Shoin, 1987); H. V. Vunakis et al. (ed.), “Methods in Enzymology”, Vol. 70 (Immunochemical Techniques, Part A), Academic Press, New York (1980); J. J. Langone et al. (ed.), “Methods in Enzymology”, Vol. 73 (Immunochemical Techniques, Part B), Academic Press, New York (1981); J. J. Langone et al. (ed.), “Methods in Enzymology”, Vol. 74 (Immunochemical Techniques, Part C), Academic Press, New York (1981); J. J. Langone et al. (ed.), “Methods in Enzymology”, Vol. 84 (Immunochemical Techniques, Part D: Selected Immunoassays), Academic Press, New York (1982); J. J. Langone et al. (ed.), “Methods in Enzymology”, Vol. 92 (Inmunochemical Techniques, Part E: Monoclonal Antibodies and General Immunoassay Methods), Academic Press, New York (1983); J. J. Langone et al. (ed.), “Methods in Enzymology”, Vol. 121 (Immunochemical Techniques, Part I: Hybridoma Technology and Monoclonal Antibodies), Academic Press, New York (1986); J. J. Langone et al. (ed.), “Methods in Enzymolgy”, Vol. 178 (Antibodies, Antigens, and Molecular Mimicry), Academic Press, New York (1989); M. Wilchek et al. (ed.), “Methods in Enzymology”, Vol. 184 (Avidin-Biotin Technology), Academic Press, New York (1990); J. J. Langone et al. (ed.), “Methods in Enzymology”, Vol. 203 (Molecular Design and Modeling: Concepts and Applications, Part B; Antibodies and Antigens, Nucleic Acids, Polysaccharides, and Drugs), Academic Press, New York (1991) and the like, or the description in the references cited therein).

This application provides a diagnostic method in which the protein level in a cell extract or blood is measured in such a solid phase system as the invention (7). In other words, this invention (7) is a method of diagnosing a disease related to endometriosis characterized by comprising at least the following steps of:
(a) contacting a biological sample isolated from a subject with a support on which the foregoing antibody of the invention (2) has been immobilized;
(b) washing the support with which the biological sample has been brought into contact in the step (a);
(c) contacting the foregoing antibody of the invention (3), which has been labeled, with the support washed in the step (b);
(d) measuring a bound label or a free label on the support;
(e) comparing the label amount measured in the step (d), as an indicator of the HRF protein level, with the result of a normal biological sample; and
(f) employing a significantly higher HRF protein level compared with that of the normal biological sample as an indicator showing a disease related to endometriosis or the degree of its risk.

In addition, a diagnostic kit of the invention (11) is provided as the one capable of performing such a diagnostic method conveniently and in a wide range.

Further, this application provides the diagnostic method of the invention (8) as a method of measuring the HRF protein level in a tissue or a cell in a solid phase system. In other words, this method is a method of diagnosing a disease related to endometriosis characterized by comprising at least the following steps of:
(a) subjecting a biological sample from a subject to a treatment of tissue fixation;
(b) sectioning the fixed tissue specimen prepared in the step (a);
(c) subjecting the sectioned tissue obtained in the step (b) to immunohistological staining with the foregoing antibody of the invention (2);
(d) comparing the degree of the immunohistological staining by the step (c), as an indicator of the HRF protein level, with the result of a normal biological sample; and
(e) employing a significantly higher HRF protein level compared with that of the normal biological sample as an indicator showing a disease related to endometriosis or the degree of its risk.

In this method of the invention (8), immunohistological staining by an antibody may be carried out with one type of antibody, or with 2 types of antibodies (e.g., an antibody of the invention (2) and an labeled anti-Ig antibody or the like).

As a means for efficiently making such a diagnosis of the invention (8) or the like, the diagnostic kit of the invention (9) is provided.

The diagnostic kits of the inventions (9) to (11) are reagent kits for performing each of the foregoing diagnostic methods. With regard to such a kit, various types of kits are commercially available depending on the types of components to be tested. The diagnostic kits of this invention can be constituted by all sorts of elements to be used in a known and publicly used kit except for using an antibody and/or a labeled antibody provided by this invention.

Incidentally, the diagnostic method provided by this application can be carried out in combination with 2 or more of the foregoing methods, or can be used together with, for example, a method of measuring the expression level of a gene encoding the HRF protein by a known method (e.g., Northern blotting method, RT-PCR method, DNA microarray method or the like).

The invention (12) is an antibody recognizing an HRF protein and neutralizing the activity of the HRF protein, and the invention (13) is a therapeutic drug for a disease related to endometriosis comprising this antibody. In addition, the invention (14) is a therapeutic method for a disease related to endometriosis characterized by administering the foregoing antibody or therapeutic drug into the body.

The antibody of the invention (12) is an antibody neutralizing the activity of the HRF protein (i.e., inhibiting or suppressing the activity of the HRF protein), and is effective for treating a disease related to endometriosis. As shown in the Examples described later, since a cell producing an excessive amount of HRF protein proliferates actively in vivo, it is considered that the excessive activity of the HRF protein in a cell causes transplantation or proliferation of an endometrial tissue. Therefore, by neutralizing the activity of the HRF protein, it is possible to treat the disease related to endometriosis. or at least arrest or suppress its progress or aggravation.

As a result of injecting a cell in which HRF has been forcibly expressed into the body of an animal such as mouse, for example, into the abdominal cavity thereof, endometriosis-like lesion is caused in the abdominal cavity. Therefore, it is evident that, by utilizing the foregoing antibody capable of neutralizing HRF, the effect of HRF is suppressed or the like, whereby it can be used for therapy.

The antibody of the invention (12) is preferably a monoclonal antibody, more preferably a humanized monoclonal antibody. A method of humanizing a non-human antibody is well known, and a humanized monoclonal antibody can be prepared by, for example, replacing the complementarity-determining region (CDR) of an antibody derived from a rodent with a corresponding sequence of a human antibody (e.g., Jones et al., Nature, 1986, 321: 522-525; Riechmann et al., Nature, 1988, 322: 323-327; Verhoeyen et al., Science, 1988, 239: 1534-1536). Such a “humanized” antibody is a chimeric antibody which has an intact human variable domain and in which one or more amino acid residues have been replaced with a corresponding sequence derived from a non-human species (e.g., U.S. Pat. No. 4,816,567). In fact, a humanized antibody is an antibody in which typically several CDR residues and in some situations, several FR residues have been replaced with residues from a corresponding site of a rodent antibody. In addition, a humanized antibody can be prepared in accordance with several known methods (e.g., Hoogenboom and Winter, 1991, J. Mol. Biol., 227: 381; Marks et al., 1991, J. Mol. Biol., 222: 581; Cole et al., 1985, Monoclonal Antibodies and Cancer Therapy, Alan R. Liss. p. 77; Boerner et al., 1991, J. Immunol., 147(1): 86-95) other than this. In addition, a humanized antibody can be produced by introducing a human immunoglobulin locus into a transgenic animal, for example, a mouse whose endogenous immunoglobulin gene has been partially or completely inactivated (e.g., U.S. Pat. Nos. 5,545,807, 5,545,806, 5,569,825, 5,625,126, 5,633,425, 5,661,016, Marks et al., 1992, Bio/Technology 10, 779-783; Lonberg et al., 1994, Nature, 368: 856-859; Morrison, 1994, Nature, 368: 812-13; Fishwild et al., 1996, Nature Biotechnology, 14: 845-51; Neuberger, 1996, Nature Biotechnology, 14: 826; Lonberg and Huszar, 1995, Intern. Rev. Immunol., 13: 65-93).

The drug of the invention (13) is formulated as the one containing the foregoing antibody. In other words, it is prepared for storage by mixing the antibody with a desired degree of purity with an optional pharmaceutically acceptable carrier, excipient or stabilizer in the form of a lipophilic preparation or an aqueous solution (Remington's Pharmaceutical Science 18th edition 1990). The acceptable carrier, excipient or stabilizer can be appropriately selected depending on the dosage form or administration route with the proviso that it is not toxic to a patient at the dose and concentration to be used. Examples thereof include buffers such as phosphate, citrate and other organic acids; antioxidants including ascorbic acid and methionine; preservatives (octadecyl dimethylbenzyl ammonium chloride, hexamethonium chloride, benzalconium chloride, benzethonium chloride, phenol, butyl or benzyl alcohol, alkyl paraben such as methyl or propyl paraben; catechol; resorcinol; cyclohexanol; 3-pentanol; m-cresol; etc.); low molecular weight (less than about 10 residues) polypeptides; proteins such as serum albumin, gelatin and immunoglobulin; hydrophilic polymers such as polyvinyl pyrrolidone; amino acids such as glycine, glutamine, asparagine, histidine, arginine and lysine; monosaccharides, disaccharides and other carbohydrates including glucose, mannose, and dextrins; chelating agents such as EDTA; sugars such as sucrose, mannitol, trehalose and sorbitol; salt-forming counterions such as sodium; metal complexes (e.g., Zn-protein complex); nonionic surfactants such as Tween (trade name), Pluronics (trade name), and polyethylene glycol (PEG), and the like.

The drug of this invention may also contain an active component such as a cytotoxic agent, cytokine or growth inhibitory agent. Such a component may also be incorporated in a microcapsule prepared, for example, by a coacervation technique or by interfacial polymerization, [e.g., hydroxymethylcellulose or gelatin-microcapsule and poly-(methylmethacylate) microcapsule, respectively] or in a colloidal drug delivery system (e.g., a liposome, albumin microsphers, microemulsion, nano-particle and nanocapsule) (see Remington's Pharmaceutical Sciences 18th edition, 1990).

Further, the preparation to be administered into the body has to be aseptic. This is easily accomplished by filtration through a sterile filtration membrane. A sustained-release preparation may be prepared. Suitable examples of the sustained-release preparation include semipermeable matrices of solid hydrophobic polymers containing the antibody (in the form of, for example, a film, or microcapsule). Examples of the sustained-release matrices include polyester hydrogels (e.g., poly(2-hydroxyethyl-methacrylate), and poly(vinylalcohol), polylactides (U.S. Pat. No. 3,773,919), L-glutamic acid and γ-ethyl-L-glutamate, non-degradable ethylene-vinyl acetate, degradable lactic acid-glycolic acid copolymers such as the LUPRON DEPOT (trade name) (injectable microspheres composed of lactic acid-glycolic acid copolymer and leuprolide acetate), poly-D-3-hydroxybutyric acid and the like. In addition, polymers such as ethylene-vinyl acetate and lactic acid-glycolic acid enable release of molecules for over 100 days.

The therapeutic method of the invention (14) can be carried out by administering the foregoing antibody or drug into the body. For example, it is a method of locally administrating the drug into endometrium or systemically administering it through vein, or the like. The administration amount of the antibody can be set in a range from about 100 μg/Kg of body weight to about 10 mg/Kg of body weight depending on the body weight and symptoms of a patient and the like.

### EXAMPLES

Hereunder, the invention of this application will be described in more detail and specifically with reference to Examples, however, the invention of this application is not limited to the following examples.

### Example 1

1. Materials and Methods

1-1. Tissue Samples

In order to prepare RNA, the following samples were obtained from 18 cases of patients: 1) endometriotic implants (n=21), 2) eutopic endometrial tissues derived from a patient with endometriosis (by curettage; n=4), and 3) normal endometrial tissues derived from a patient without endometriosis (n=6). Several samples were obtained from different sites of one individual. The samples were freezed in liquid nitrogen, and stored at −80° C. for RNA preparation. The endometriotic implants were obtained from an ovary. The samples obtained by formalin fixation and paraffin embedding of the normal endometrial tissues for RNA preparation and endometrial tissues that normally proliferate and make a secretion were obtained from a patient with leiomyoma or uterine prolapse. As a result of grading the pathologic specimens by a histological study, they ranged from stage III to stage IV of endometriosis (t-ASRM: revised American Society for Reproductive Medicine classification of endometriosis, 1996). In addition, the female subjects of this study did not show endometrial hyperplasia or tumor formation, nor did they receive the administeration of an anti-inflammatory agent or hormonal agent before surgery. Before surgery, a written consent was obtained, which was carried out in accordance with the protocol approved by the internal audit committee with regard to the investigation of the human body at Tokyo Medical University Hospital.

1-2. Northern Blot Analysis

Northern blot was carried out in accordance with the description in the literature (Oikawa K. et al., Cancer Res. 2001, 61(15) 5707-9). An HRF probe was prepared in accordance with the description in the literature (Oikawa K. et al., Biochem. Biophys. Res. Commun. 2002, 290(3): 984-7). A human β-actin cDNA control probe (CLONTECH Laboratories Inc.) was used as a standard.

1-3. RT-PCR Using Southern Blot Analysis

First-strand cDNA synthesis from total RNA was carried out by using an oligonucleotide dT primer in accordance with the description in the literature (Kubota M. et al., Am. J. Pathol. 1997, 151(3): 735-44). Then, PCR was carried out by using 2 μl (1×) and 10 μl (5×) of a solution of the obtained first-strand cDNA as a template. After the following 4 types of primers were added, PCR amplification of cDNA fragments of CYP1A1 and β-actin was carried out under the condition of initial denaturation at 95° C. for 2 minutes followed by 22 cycles of 95° C. for 0.5 minutes, 65° C. for 0.5 minutes and 72° C. for 1 minute.

Primer for CYP1A1 amplification:

5′-ccacaaccaccaagaactgcttag-3′(SEQ ID: 3)

5′-gaaggggacgaaggaagagtg-3′(SEQ ID: 4)

Primer for β-actin Amplification:

5′-gggaaatcgtgcgtgacgttaag-3′(SEQ ID: 5)

5′-tgtgttggcgtacaggtctttg-3′(SEQ ID: 6)

After the amplified products were fractionated on an agarose gel by electrophoresis, blotting and hybridization were carried out. The CYP1A1 cDNA probe was obtained by reverse transcription PCR with the foregoing primer pair. The human β-actin cDNA probe (CLONTECH) was used as a control. By using Rediprime II random trime labeling system (Amersham Pharmacia Biotech), these cDNA probes were labeled with ³²P.

1-4. Preparation of Antibody and Immunohistochemical Method

A peptide antibody against an oligopeptide (GKLEEQRPERVKPFMT: at positions 101 to 116 of SEQ ID: 2) derived from human HRF was prepared by a standard method using a rabbit and named HRF-GKL. With regard to immunohistochemical analysis, deparaffinized sections were incubated overnight with a mixed solution of an anti-HRF antibody, HRF-TPY (Oikawa K. et al., Biochem. Biophys, Res. Commun. 2002, 290(3): 984-7) and HRF-GKL (diluted to 1:100) or an anti-human CD68 antibody (diluted to 1:100; Dako Inc). For anti-HRF staining, deparaffinized sections were subjected to heat-induced antigen recovery with a pressure sterilizer. Detection was carried out using LSABC (Dako), in which 3,3-diaminobenzidine was used as a plastid. Reverse staining was carried out using hematoxylin.

1-5. Western Blot Analysis

Western blot analysis was carried out in accordance with the description in the literature (Oikawa K. et al., Biochem. Biophys, Res. Commun. 2002, 290(3): 984-7). The membrane was probed with an anti-HRF (HRF-GKL or HRF-TPY) antibody at a dilution ratio of 1:2000. Signal detection was carried out using ECL plus Western blotting detection system (Amersham Pharmacia Biotech).

1-6. Cell Culture and Retrovirus Infection

NIH3T3 cell was obtained from American Type Culture Collection (ATCC). The cell was maintained at 37° C. under a 5% CO₂ atmosphere in DMEM (GIBCO BRL, Life Technologies, Inc.) to which 10% FBS was added. A mouse HRF cDNA containing the full-length ORF was amplified by PCR using the following primers.

5′-ttggatccatgatcatctaccgggacctg-3′(SEQ ID: 7)

5′-ttgaattcttaacatttctccatctctaa-3′(SEQ ID: 8)

The obtained cDNA fragment was digested with BamHI and EcoRI, and cloned at the BgIII-EcoRI site of a retrovirus expression vector, MSCV-puro (CLONTECH). The protocol for preparation and infection of a recombinant retrovirus was carried out in accordance with the description of the literature (Kuroda> et al., Proc. Natl. Acad. Sci. USA 1999, 96(9): 5025-30). Twenty-four hours after the infection, by using 1 μg/ml of puromycin (CLONTECH), an infected cell was selected for over 2 weeks.

1-7. Animals and Treatment

Transplantation assay was carried out by injecting a partial specimen of 5×10⁵ cells into the abdominal-cavity of a female BALB/C nude mouse at 6 weeks of age. After 2 weeks, the animal was killed, and the number of transplanted colonies was counted.

2. Results

2-1. Expression Pattern of TCDD Induced Gene HRF in Endometriosis

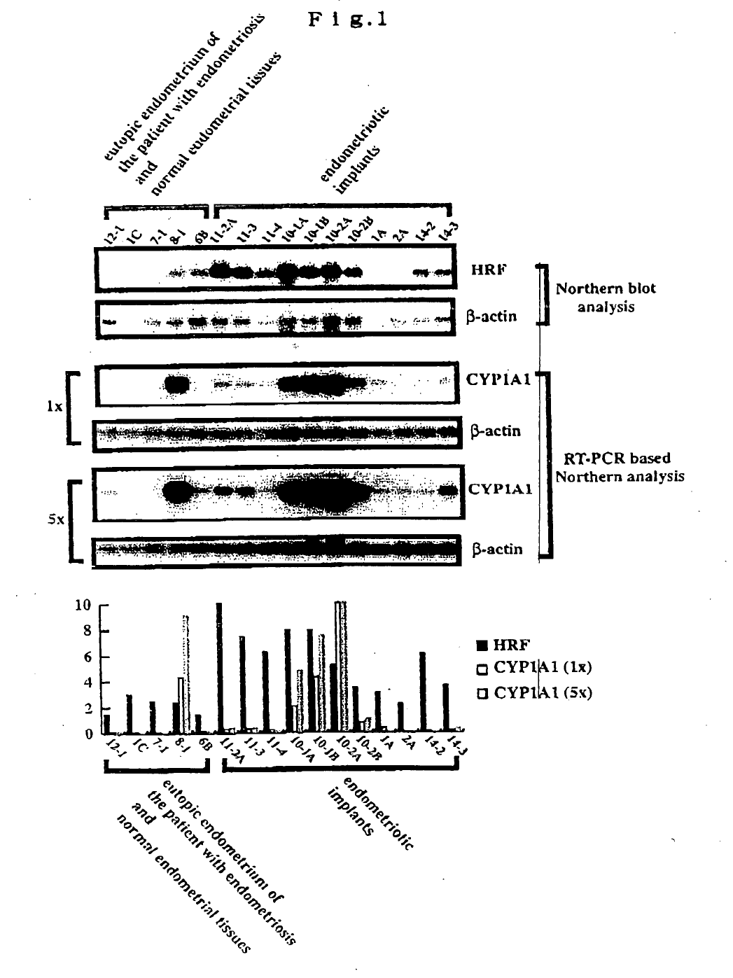

By Northern blot analysis, an HRF expression pattern during endometriosis was determined. As a result, a high level of HRF expression was confirmed in the endometriotic implant tissues obtained from 3 cases of patients out of 5 cases (FIGS. 1A and 1B). Some of the human cytochrome P450 gene superfamily (e.g., CYP1A1, CYP1A2 and CYP1B1) are induced by dioxin, therefore, induction of CYP1A1 becomes a basic target for dioxin-dependent gene expression regulation. For this reason, in order to investigate the correlation between dioxin exposure and HRF expression, CYP1A1 expression was investigated using RT-PCR by Southern analysis here (Trifa Y. et al., J. Biol. Chem. 1998, 273(7): 3980-5; Oikawa K. et al., Gene 2000, 261(2): 221-8). As a result, CYP1A1 was not always induced in all the cases showing a high HRF expression (FIGS. 1A and 1B). Thus, it was confirmed that HRF in endometriotic implants was induced regardless of TCDD exposure, though there is a possibility that HRF expression may be induced by TCDD in some cases.

2-2. Overexpression of HRF in Endometriotic Implants

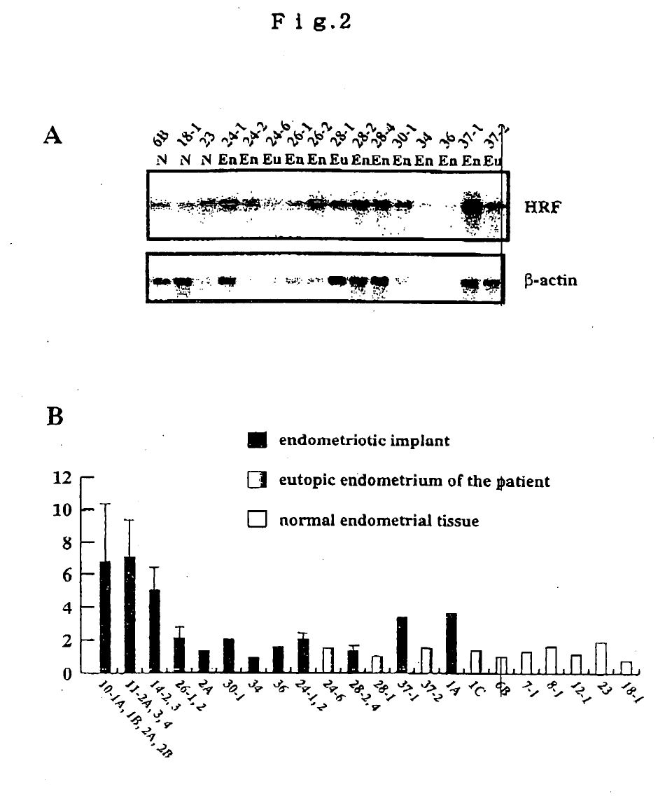

It was confirmed that HRF was overexpressed in the endometriotic implants of a patient who additionally developed endometriosis. That is, with regard to 7 cases of patients with endometriosis, Northern blot analysis was carried out (FIG. 2A). Compared with the normal endometrial tissues and the eutopic endometrium of a patient with endometriosis, a high degree of HRF expression was observed in the endometriotic implants (FIG. 2B).

2-3. Immunohistochemistry of HRF in Normal Endometrium and Endometriotic Implant

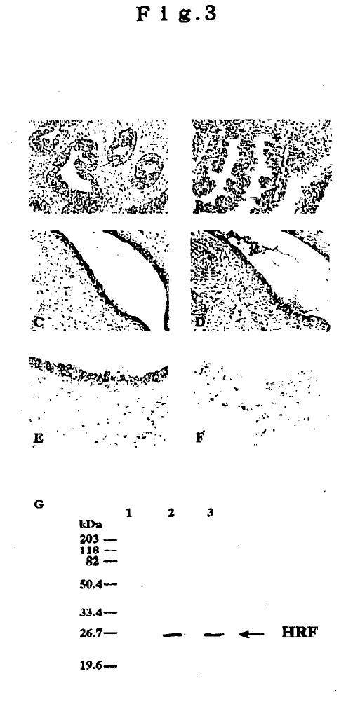

The type of endometrial cell that expresses HRF was determined by immunohistochemistry using an anti-HRF polyclonal antibody. As a result, it was identified that HRF was present in both an endometrial gland and an interstitial cell of a normal tissue, however, stronger expression was observed in the endometrial gland than the interstitial cell (FIGS. 3A and 3B). There was no significant change in the expression patterns between secretory phase and proliferative phase. Further, HRF expression was investigated in an endometriotic implant. As a result, HRF was present in both interstitial and epithelial components of an ovarian endometriotic implant (FIGS. 3C and 3E). While HRF expression was weak in the interstitial cell of normal endometrium, a similarly high level of HRF expression was observed in both the endometrial gland and the interstitial cell of the ovarian endometriotic implant. These signals specific to HRF were not observed in the case of using preimmune serum as a control (data not shown). However, the mechanism of HRF induction in an endometriotic implant remains unknown. As it agrees with the report (Teshima S. et al., J. Immunol. 1998, 161(11): 6353-66), which shows that a macrophage induces HRF at the activation stage by M-CSF, involvement of CD68 positive macrophage was observed in an endometriotic implant (Hormung D. et al., Am. J. Pathol. 2001, 158(6): 1949-54). Therefore, by utilizing CD68 staining for a series of sections of the implant, CD68 positive macrophage in the region overexpressing HRF was identified (FIG. 3F). Control sections stained with hematoxylin-eosin indicate the overall pattern of an endometriotic implant. These results suggest that a macrophage contributes to HRF production in an endometriotic implant.

2-4. Effect of HRF on Intraperitoneal Transplantation of NIH3T3 Cell

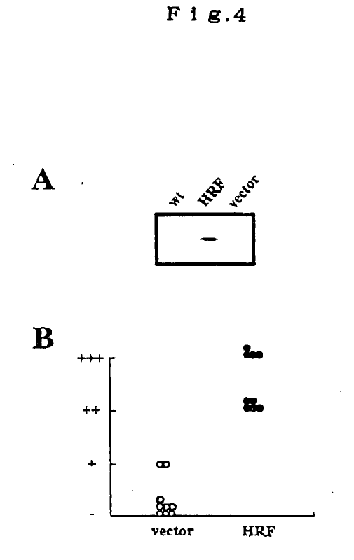

A physiological effect of the increase in HRF expression was investigated. The cause of endometriosis remains unknown (Klninckx R. P. et al., Gynecol Obstet Invest. 1999, 47 Suppl 1: 3-9, discussion 9-10; van dcr Linden P. J. Q. Front Biosci. 1997, 2: c48-52). If a major assumption is followed, the onset of endometriosis is caused by transplantation and proliferation of an endometrial tissue which enters the abdominal cavity through the fallopian tubes via reflux (retrograde menstruation). Here, an effect of HRF on the transplantation was investigated. First, a stable transfectant of NIH3T3 cell that overexpresses HRF was prepared. After the cells were infected with a retrovirus vector for HRF expression (pMSCV-HRF), a high degree of HRF expression was confirmed (FIG. 4A). Then, these cells (pMSCV-HRF-3T3 cells) were injected into the abdominal cavity of a nude mouse. The pMSCV-HRF-3T3 cell had a higher transplantability compared with a cell infected with a control vector (pMSC-3T3) (FIG. 4B). These data suggested that HRF plays an important role not only in immunological dysfunction but also in initial development of an endometriotic implant.

### Example 2

### Preparation of Polyclonal Antibody

An antiserum containing an anti-HRF antibody was prepared, and the anti-HRF antibody was purified from this serum.

As a sensitizing antigen, the peptide at positions 101 to 116 of the human HRF protein (SEQ ID NO: 2) was selected and synthesized. The synthesized peptide was bound to HLA, as a carrier, and mixed with KLH (50 μg of KLH relative to 50 μg of the peptide). The thus obtained antigen solution was mixed with Freund's complete adjuvant, whereby a solution containing the sensitizing antigen was prepared. This solution was subcutaneously injected into a rabbit (SPF Japanese White Rabbit) weighing 3 to 4 kg in an amount of 1 ml every two weeks (5 times).

Then, one week after the 5th subcutaneous injection, blood was collected from the rabbit, and serum was prepared. It was confirmed that the antibody contained in the obtained serum specifically recognized an HRF protein, which was used as an antiserum containing an anti-HRF antibody.

By the ELISA method, it was confirmed that the antiserum contains an anti-HRF antibody (HRF-GKL).

First, a 96-well plate made of polystyrene was coated with the antigen polypeptide that was diluted with 20 mM carbonate buffer (pH 9.6) (100 ng/well). Then, the plate was washed with PBS containing 0.05% Tween 20 thereby removing unadsorbed peptides. To each well, the serum obtained by collecting blood from the immunized rabbit is added, and the plate is let stand at room temperature for about 1 hour. After washing, horseradish peroxidase (HRP)-labeled anti-rabbit immunoglobulin is added as a secondary antibody, and the plate is further let stand at room temperature for about 1 hour. After washing, hydrogen peroxide, which is a substrate, and 3,3′,5,5′-tetramethylbenzidine (TMB) are added to develop color. To each well, 2 N sulfuric acid is added to terminate the coloring reaction, and the degree of coloring is measured at 450 nm of optical density with an optical density measuring device for microplate. By way of comparison, it is compared with that of the serum obtained from the blood collected before immunization, whereby it is confirmed that the antibody contained in the serum surely recognizes the HRF protein specifically. The foregoing procedure can be also carried out by using a recombinant protein antigen.

With regard to the obtained antiserum containing the anti-HRF antibody, its purity is confirmed by loading 10 μg of total cell lysate of NIH3T3 on a 14% SDS-PAGE, and performing an assay by Western blot with the antiserum diluted to 2,000-fold thereby providing a single band. This antibody was an antibody that detects proteins of the same size in mouse and human cells.

A purified anti-HRF antibody can be prepared by affinity chromatography with a column in which the foregoing peptide at positions 101 to 116 of the HRF protein has been immobilized on Affigel (manufactured by BioRad Inc). The purified HRF peptide is mixed with Affigel-10 (manufactured by BioRad Inc.), and they are reacted overnight at 4° C. Then, the Affigel was thoroughly washed with 20 mM phosphate buffer-saline (PBS), and blocking of an unreacted functional group of the Affigel is carried out overnight in PBS containing 100 mM monoethanolamine. Finally, the Affigel was washed with PBS again, whereby a peptide-immobilized column is prepared. To this immobilized column, the rabbit serum containing the anti-HRF antibody is added, and the column was thoroughly washed with PBS. Then, the adsorbed anti-HRF antibody is eluted with 20 mM glycine-HCl buffer (pH 4.0). The solution containing the eluted anti-HRF antibody is immediately neutralized with 200 mM Tris-HCl buffer, dialyzed overnight with PBS, and cryopreserved at −80° C.

### Example 3

### Sandwich EIA

According to the following method, a sandwich EIA system of specifically detecting and measuring a human HRF protein can be constructed by selecting at least one type from the anti-HRF antibody prepared in Example 2 and known anti-HRF antibodies and appropriately combining 2 types of anti-HRF antibodies. It is possible to employ either a one-step EIA system or a two-step EIA system. A labeled antibody is not limited to Fab′-HRP. The composition of each reaction buffer or the reaction condition can be adjusted (such as shortening or extending) depending on the purpose of the measurement. In addition, human HRF, which becomes a standard, can be purified from a tissue culture supernatant, a cell culture supernatant or a recombinant expressed by the method described in Example 1 or another method. The purification is attained by ion exchange chromatography, get filtration, affinity chromatography using an anti-human HRF antibody or by a combination of a variety of chromatographies other than these.

(a) Preparation of Labeled Antibody

To 0.1 M acetate buffer (pH 4.2) containing 0.1 M NaCl and an anti-HRF antibody (HRF-GKL), pepsin in an amount of 2% (W/W) of the amount of the antibody is added, and digestion is carried out at 37° C. for 24 hours. To the digested solution, 3 M Tris-HCl (pH 7.5) is added to terminate the reaction. By gel filtration using Ultrogel AcA54 column equilibrated with 0.1 M phosphate buffer (pH 7.0), F(ab′)₂ fraction is fractionated. To this F(ab′)₂ fraction, cysteamine hydrochloride is added at a final concentration of 0.01 M, and reduction is carried out at 37° C. for 1.5 hours. Then, by gel filtration using Ultrogel AcA54 column equilibrated with 0.1 M phosphate buffer (pH 6.0) containing 5 mM DETA, Fab′ fraction is fractionated.

Aside from the foregoing procedure, HRP is dissolved in (0.1 M phosphate buffer (pH 7.0), and EMCS in an amount of 25 times the molar amount of HRP is added as a DMF solution, and they are reacted at 30° C. for 30 minutes. This solution is subjected to gel filtration using NICK-5 column (Pharmacia) equilibrated with 0.1 M phosphate buffer (pH 6.0), whereby maleimide-labeled HRP fraction is fractionated.

The Fab′ fraction and the maleimide-labeled HRP fraction were mixed so that the solution contains equivalent amounts of Fab′ and maleimide-labeled HRP, and they are reacted at 4° C. for 20 hours. Then, an unreacted thiol group is blocked with N-ethylmaleimide in an amount of 10 times the molar amount of Fab′. This solution is subjected to gel filtration using Ultrogel AcA54 column equilibrated with 0.1 M phosphate buffer (pH 6.5), whereby a Fab′-HRP-labeled antibody is fractionated. Thereto are added 0.1% BSA and 0.001% chlorhexidine, and the mixture is stored at 4° C. It can be treated in the same way using another anti-human HRF antibody.

(b) Preparation of Antibody-bound Support

An anti-HRF antibody (HRF-TPY) is dissolved at a concentration of 50 μg/mL in 0.1 M phosphate buffer (pH 7.5). This antibody solution was added to a 96-well microplate in an amount of 100 μL per each well, and let stand at 4° C. for 18 hours. The antibody solution was removed, and the plate was washed with saline once and with Tris-HCl buffer (pH 8.0) containing 0.05% Tween 20, 0.1 M NaCl and 5 mM CaCl₂ 3 times. Then blocking is carried out by adding Tris-HCl buffer (pH 8.0) containing 1% BSA, 0.1 M NaCl anid 5 mM CaCl₂. It can be treated in the same way using another anti-human HRF antibody, and a solid phase antibody can be prepared.

(c) Step Sandwich EIA Method

A standard curve for quantification of human HRF is constructed by using the purified human HRF fraction as a standard antigen. The standard human HRF serially diluted with Tris-HCl buffer (pH 8.0) containing 1% BSA, 0.05% Tween 20, 0.1 M NaCl and 5 mM CaCl₂ is dispensed, and the labeled antibody Fab′-HRP prepared with Tris-HCl buffer (pH 8.0) containing 1% BSA, 0.05% Tween 20, 0.1 M NaCl and 5 mM CaCl₂ is added to each well and mixed throughly. The prepared antibody-bound microplate is washed 3 times with Tris-HCl buffer (pH 8.0) containing 0.05% Tween 20, 0.1 M NaCl and 5 mM CaCl₂, and a mixed solution of the standard antigen and the standard antibody is added. After they are reacted at room temperature for 1 hour, the plate is washed 3 times with Tris-HCl buffer (pH 8.0) containing 0.05% Tween 20, 0.1 M NaCl and 5 mM CaCl₂. Then, 0.01% 3,3′,5,5′-tetramethylbenzidine dissolved in 0.1 M acetate buffer (pH 5.5) containing 6% dimethylformamide and 0.005% hydrogen peroxide is added to each well, and they are reacted at room temperature for 20 minutes. Then, 2 N sulfuric acid is added to terminate the reaction. The optical density at 450 nm of this reaction mixture is measured with a microplate reader, and a standard curve is obtained.

A measured sample is prepared from a body fluid component derived from human, an extract from a variety of human tissues, a cell extract from a variety of culture cells such as a cell derived from human or a recombinant, culture supernatant or the like. Each measured sample is subjected to the foregoing one-step sandwich EIA instead of the standard human HRF, and the reaction is allowed to proceed simultaneously with the standard human HRF. The optical density obtained from the measured sample is applied to the standard curve to calculate the amount of human HRF contained in the measured sample.

Other than the above, an enzyme-labeled antibody can be prepared by using a commercially available anti-human HRF antibody in accordance with the methods described in Ikuzo Uritani et al. ed., “Seitutsu Kagaku Jikkenho 27, written by Eiji Ishikawa, Koso Hyoshikiho”, Gakkai Syuppan Center (published on Jun. 20, 1991) and Japanese Biochemical Society ed., “Zoku Seikagaku Jikken Koza 5, Meneki Seikagaku Kenkyuho” pp. 107-112, Tokyo Kagaku Dozin, published on Mar. 14, 1986 (including the methods described in the references cited in these literatures), and further it can be used in measurement.

### Example 4

### Western Blotting

After the culture supernatant of cells or tissues, which express human HRF and the purified recombinant human HRF were separated on 10 to 15% SDS-PAGE under reduction conditions, they were transferred to polyvinylidene difluoride (PVDF) membranes (MILLIPORE). Then, blocking was carried out at room temperature for 0.5 to 1 hour by using TBS containing 5% BSA or 5% skim milk and 0.05% sodium azide (blocking buffer). Then, the membranes are treated with HRF-GKL, and incubated at 25° C. for 6 hours or less. The respective membranes are washed 4 times with TBS (containing 0.05% sodium azide) containing (0.1% Tween 20, and the bound antibody is reacted with an HRP-conjugated anti-rabbit immunoglobulin antibody diluted to 1:1,000 with the blocking buffer at 25° C. for 1 hour. After the reaction, the membranes are washed 4 times with TBS (containing 0.05%/o sodium azide) containing 0.1% Tween 20, and the bound antibody was detected by Enhanced chemiluminescence (ECL, Amersham Pharmacia).

Other than the above, Western blotting can be carried out by using HRF-TPY in accordance with the methods described in Yoshiyuki Kuchino et al., ed., “Gene/Protein, Experimental Procedure, Blotting Method” pp. 212-241, Soft Science Inc., published on Nov. 10, 1987 (including the methods described in the references cited in these literatures).

### Example 5

### Preparation of Monoclonal Antibody

A full-length human HRF cDNA was isolated by the TR-PCR method using the total RNA prepared from cells in human endometriotic lesion tissues collected from a patient. As a PCR primer, based on the full-length sequence (SEQ ID; 1) of the human HRF cDNA, the following oligonucleotides were used.

(SEQ ID: 9)

5′ primer (BglII) gcgcagatctATGATTATCTACCGGGAC

(SEQ ID: 10)

3′ primer (EcoRI) ggccgaattcAGATCCAAAATAATTGCC

Then, the obtained PCR product was cloned into a human baculovirus vector, pYNG HisA, and a silk worm was infected with the vector. Then, the human HRF protein was extracted from the body fluid of the silk worm. Then, by using a nickel column, the protein was further purified by a histidine tag. Then, a female Balb/C mouse at 6 weeks of age was immunized by injection with 100 μg of the purified protein 3 times. Then, the inguinal lymph node was excised, and fused with a mouse myeloma cell, P3U1, whereby a hybridoma was obtained. The hybridoma cells producing the HRF antibody were subjected to limiting dilution twice, and eventually, hybridomas, HRF25 HRF26, HRF28 were obtained. Screening of the hybridomas was carried out by the ELISA method. Specifically, 1 μg/ml of an antigen (the HRF protein prepared by a baculovirus) was adsorbed onto ERISA plate 3912 (FALCON, reacted with the hybridoma supernatant, and further reacted with Goat-antiMouse IgG (Zymed 81-6522) as a secondary antibody. ALP rose (SHINO-TEST) was added as a substrate, and the optical density at A660 nm was measured. As a result, clones No. 4, 18, 25, 26, 28, 46, 51, 54, 55 and 56 were obtained. The results of the measured optical density are shown in Table 1.

TABLE 1

| Clone No. | OD |
|---|---|
| 4 | 0.15 |
| 18 | 0.55 |
| 25 | 0.08 |
| 26 | 0.57 |
| 28 | 0.29 |
| 46 | 0.11 |
| 51 | 0.56 |
| 54 | 0.74 |
| 55 | 0.43 |
| 56 | 0.86 |

Further, Western blotting was carried out using these culture supernatants. Specifically, total protein was extracted from BJAB in which expression of HRF had been confirmed, SDS-PAGE was carried out in accordance with the method of Laemmili, and blotting was carried out on a nitrocellulose membrane. Then, they were reacted with a hybridoma supernatant diluted to 5-fold as a primary antibody, and investigation was carried out. As a result, it was confirmed that, with regard to the clones No. 25 (HRF25), No. 26 (HRF26) and No. 28 (HRF28), the HRF protein was detected as a single band. Incidentally, these 3 antibodies are IgG antibodies.

In addition, the foregoing results indicate that the anti-HRF antibody recognizes human HRF very specifically.

By using NIH3T3 cell, which is a cell forcibly expressing HRF described in 2-4 of Example 1, screening of the activity of the monoclonal antibody obtained above can be carried out in vivo as well as in vitro. It is judged that the antibody whose activity has been confirmed is promising in a diagnostic agent and/or a therapeutic drug for endometriosis.

### INDUSTRIAL APPLICABILITY

As described in detail above, by the invention of this application; a method of conveniently and surely diagnosing a disease related to endometriosis and its risk and a material for the method are provided. Accordingly, it becomes possible to detect a disease related to endometriosis at an early stage, to select a more appropriate therapeutic method, to prevent the recurrence thereof or the like.

1. A method of diagnosing a disease related to endometriosis, which comprises measuring the level of a histamine-releasing factor (HRF protein) in a biological sample from a subject, comparing the HRF protein level with that of a normal biological sample and determining that the subject showing a significantly higher HRF protein level compared with that of the normal biological sample is a patient with a disease related to endometriosis or a person with high risk thereof.

2. An antibody recognizing an HRF protein.

3. An antibody binding to an epitope different from the one to which an antibody of claim 2 binds.

4. The antibody of claim 2 or 3, obtained by using, as an immunizing antigen, a peptide containing a sequence of 5 to 20 amino acid residues selected from the amino acid sequence at positions 90 to 130 of SEQ ID NO: 2.

5. The antibody of claim 2 or 3, obtained by using, as an immunizing antigen, a peptide containing a sequence of 5 to 20 amino acid residues selected from the amino acid sequence at positions 1 to 95 of SEQ ID NO: 2.

6. The antibody of claim 2 or 3, obtained by using, as an immunizing antigen, a peptide containing a sequence of 5 to 20 amino acid residues selected from the amino acid sequence at positions 115 to 172 of SEQ ID NO: 2.

7. A method of diagnosing a disease related to endometriosis, which comprises at least the following steps of:
(a) contacting a biological sample from a subject with a support on which the antibody of claim 2 has been immobilized;
(b) washing the support with which the biological sample has been contacted in the step (a);
(c) contacting the antibody of claim 3, which has been labeled, with the support washed in the step (b);
(d) measuring a bound label or a free label on the support;
(e) comparing the label amount measured in the step (d), as an indicator of the HRF protein level, with the result of a normal biological sample; and
(f) employing a significantly higher HRF protein level compared with that of the normal biological sample as an indicator showing a disease related to endometriosis or the degree of its risk.

8. A method of diagnosing a disease related to endometriosis, which comprises at least the following steps of:
(a) subjecting a biological sample from a subject to a treatment of tissue fixation;
(b) sectioning the fixed tissue specimen prepared in the step (a);
(c) subjecting the sectioned tissue obtained in the step (b) to immunohistological staining with the antibody of claim 2;
(d) comparing the degree of the immunohistological staining by the step (c), as an indicator of the HRF protein level, with the result of a normal biological sample; and
(e) employing a significantly higher HRF protein level compared with that of the normal biological sample as an indicator showing a disease related to endometriosis or the degree of its risk.

9. A kit for diagnosing a disease related to endometriosis comprising at least the antibody of claim 2, which has been labeled.

10. A kit for diagnosing a disease related to endometriosis comprising at least the following elements:
(a) the antibody of claim 2; and
(b) the antibody of claim 3, which has been labeled.

11. A kit for diagnosing a disease related to endometriosis comprising at least the following elements:
(a) a support on which the antibody of claim 2 has been immobilized; and
(b) an antibody of claim 3, which has been labeled.

12. An antibody recognizing an HRF protein and neutralizing the activity of the HRF protein.

13. A therapeutic drug for a disease related to endometriosis, which comprises the antibody of claim 12.

14. A therapeutic method for a disease related to endometriosis, which comprises administering the antibody of claim 12 or a therapeutic drug of claim 13 into the body.
